(19) United States
(12) Patent Application Publication
(10) Pub. No.: US 2024/0032530 A1
(43) Pub. Date: Feb. 1, 2024

(54) BLOOD SAMPLER CONTAINING ANTI-PLATELET AGENT AND WATER-SOLUBLE MATRIX MATERIAL

(71) Applicant: Radiometer Medical ApS, Brønshøj (DK)

(72) Inventors: Kristian Mdeom HANSEN, Brønshøj (DK); Thomas Steen HANSEN, Brønshøj (DK); Melanie Andrea BURKHARDT, Brønshøj (DK)

(21) Appl. No.: 18/258,403

(22) PCT Filed: Dec. 22, 2021
(86) PCT No.: PCT/EP2021/087289

§ 371 Date: Jun. 20, 2023

(30) Foreign Application Priority Data
Dec. 22, 2020 (EP) 20216444.8

(51) Int. Cl. A01N 1/02; B01L 3/00

(52) U.S. Cl. CPC A01N 1/0226; B01L 3/50825; A01N 1/0263

(57) ABSTRACT

A blood sampler is disclosed. The blood sampler contains a solid mixture comprising a compound selected from iloprost, beraprost, treprostinil, cicaprost, carbacyclin, EP 157 ((Z)-7-[(1R,2R,3R,4S)-3-[(Z)-benzhydryloxyiminomethyl]-2-bicyclo[2.2.2]oct-5-enyl]hept-5-enoic acid), FK-788 (2-[[(6R)-6-(diphenylcarbamoyloxymethyl)-6-hydroxy-7,8-dihydro-5H-naphthalen-1-yl]oxy]acetic acid), and taprostene, as well as any stereoisomers and salts thereof, wherein said compound is dispersed in a matrix of a water-soluble polymer material and/or a sugar.

### FIELD OF THE INVENTION

The present invention concerns a blood sampler comprising a prostacyclin analog. The prostacyclin analog is dispersed in a water-soluble polymer, which keeps the compound stable and allows for a sufficient dissolution and dissolution rate of the prostacyclin analog when brought into contact with a blood sample.

### BACKGROUND OF THE INVENTION

Rapid access to blood tests is a mainstay in the diagnosis and treatment of acute disease. Oxygenation status and acid-base balance are determined by arterial blood gas (BG) analysis and constitute a central part of modern evidence-based treatment algorithms in critical care. Furthermore, devices intended for critical care testing allow for assessment of e.g. electrolytes, renal function (creatinine), inflammation (C-reactive protein) and cardiac biomarkers.

The basic metabolic panel (BMP) is used to check the status of a person's kidneys and their electrolyte and acid/base balance, as well as their blood glucose level—all of which are related to a person's metabolism. It can also be used to monitor hospitalized patients and people with certain known conditions, such as hypertension and hypokalemia.

Further, the count of white blood cells (WBC) is an important biomarker for several diseases and a differential WBC count may even differentiate five different types of blood cells (“5-diff” or “5-part diff”), namely neutrophils, lymphocytes, monocytes, eosinophils and basophils. Alternatively, WBC can be differentiated into granulocytes (neutrophils, basophils and eosinophils reported as a group), lymphocytes and monocytes (“3-diff” or “3-part diff”). Each is reported as a percentage. A shift in the percentage may indicate a pathological condition.

Further, platelets (also termed thrombocytes) may be counted as another parameter. Platelets are small fragments of cells that are essential for normal blood clotting. A platelet count may be used to screen for or diagnose various diseases and conditions that can cause problems with clot formation. It may be used as part of the workup of a bleeding disorder, bone marrow disease, or excessive clotting disorder, to name just a few.

The test may be used as a monitoring tool for people with underlying conditions or undergoing treatment with drugs known to affect platelets. It may also be used to monitor those being treated for a platelet disorder to determine if therapy is effective.

However, blood samples usually have to be differently prepared for diagnostic measurements of the abovementioned parameters. For example, for BG and BMP parameter analysis, the standard anticoagulant is heparin. Heparin prevents blood coagulation, but it however does not prevent platelet (thrombocyte) activation and aggregation, which leads to platelet aggregate formation. Heparin is therefore nowadays not used for a complete blood cell count (CBC) analysis, comprising the counts of WBC, platelets, 3-diff or 5-diff, red blood cell (RBC) concentration, hematocrit, hemoglobin concentration and RBC descriptive parameters. The measured platelet count in heparinized blood would be underestimated, in particular when using state of the art automated hematology analyzers, which are not able to distinguish single platelets from aggregated platelet clots. Instead, platelet aggregates may be misclassified by hematological analyzers as leucocytes and therefore, a falsely high WBC count is obtained potentially resulting in a flawed diagnosis or flags and error messages rendering the results unusable.

Ethylenediaminetetraacetic acid (EDTA), either as di-sodium, di-potassium or tri potassium salt, is another commonly used standard anticoagulant in hematology. The use of EDTA is generally accepted to be safe and reliable for obtaining complete blood cell counts. In addition, EDTA salts are compatible, i.e. do not interfere, with standard staining protocols for blood smears. If problems with EDTA-dependent pseudothrombocytopenia occur, citrate is used as the alternative anticoagulant.

However, EDTA or citrate cannot be used for BG and BMP parameter analysis as these anticoagulants strongly interfere with electrolyte measurements. For example, EDTA and citrate form a complex with Ca2+ and thus interfere with Ca2+ measurements and these anticoagulants might even destroy the calcium-sensors of the automated analyzers.

Previously, hematology analysis, and in particular CBC, was performed on EDTA or citrate anticoagulated blood samples and not on heparinized blood samples. As a consequence, a comprehensive analysis of CBC, BG and BMP parameters needed to be performed with separate differently anticoagulated blood samples on separate instruments.

WO 2019/096598 discloses a method for preparing a blood sample combining an anti-coagulant, such as heparin, with an anti-platelet agent, such as iloprost. This combination allows for a “3-in-1” analysis of BG, BMP, and platelet count, as well as a WBC (or CBC) count.

WO 2020/229580 further discloses that such a method may be carried out even if the blood sample has been exposed to stress, such as low temperatures.

JP H01 280466 discloses the preparation of a formulation of the anti-platelet agent prostacyclin by polymerizing polyacrylamide together with prostacyclin in an organic solvent, drying the obtained particles, and then dispersing the dried particles in a polymer solution in an organic solvent and leaving to dry to obtain a film of the polymer. The polymer is not water-soluble.

Accordingly, there is a need for new approaches for preparing a blood sample for diagnostic measurements.

### SUMMARY OF THE INVENTION

It would be advantageous to be able to contain the anti-platelet agent, such as iloprost, and/or the anti-coagulant, such as heparin, in a blood sampler ready to use in methods such as those discussed above.

A number of prostacyclin analogs (also sometimes referred to as prostaglandin analogs) have anti-platelet activity. However, they are typically not very stable. As an example, iloprost is sold as an inhalation solution under the brand name Ventavis®, which is approved for the treatment of pulmonary hypertension. The European Medicines Agency characterize iloprost as being sensitive to temperature, light, and acidic conditions. The degradation products are mainly dimeric esters formed by addition of two iloprost molecules.

Accordingly, a need exists for containing prostacyclin analogs, such as iloprost, in a blood sampler in a way that prevents or delays the known degradation while also allowing sufficiently rapid dissolution of the prostacyclin analogs upon exposure to a blood sample.

This need has been met by the present invention, which in one aspect concerns a blood sampler containing a solid mixture comprising a compound selected from iloprost, beraprost, treprostinil, cicaprost, carbacyclin, EP 157 ((Z)-7-[(1R,2R,3R,4S)-3-[(Z)-benzhydryloxyiminomethyl]-2-bicyclo[2.2.2]oct-5-enyl]hept-5-enoic acid), FK-788 (2-[[(6R)-6-(diphenylcarbamoyloxymethyl)-6-hydroxy-7,8-dihydro-5H-naphthalen-1-yl]oxy]acetic acid), and taprostene, as well as any stereoisomers and salts thereof, wherein said compound is dispersed in a matrix of a water-soluble polymer and/or a sugar.

In a further aspect, the present invention concerns the use of the blood sampler according to the invention for measuring albumin, alkaline phosphatase, lactate dehydrogenase, alanine transaminase, aspartate transaminase, amylase, lipase, total cholesterol, high density lipoprotein, low density lipoprotein, triglycerides, thyroid-stimulating hormone, troponins, creatine kinase, myoglobin, D-dimer, N-terminal prohormone of brain natriuretic peptide (NT-proBNP), procalcitonin (PCT), C-reactive protein (CRP), beta-human chorionic gonadotropin, CBC, BG and BMP parameters in a blood sample.

### DETAILED DESCRIPTION OF THE INVENTION

### Definitions

In the context of the present invention, the term “blood sampler” is intended to mean a device for collection of blood, such as a syringe, a capillary tube, or a test tube, e.g. an aspirating sampler or self-aspirating sampler, such as a PICO™ syringe (Radiometer Medical ApS), a vacuum test tube or a similar device designated for blood sampling. In one embodiment, the blood sampler is a device for collection of blood. In a further embodiment, the blood sampler is selected from a syringe, a capillary, a test tube, and a cuvette. In still a further embodiment, the blood sampler is selected from a syringe, a capillary, and a test tube.

As used herein, the term “anticoagulant” means a substance that prevents or reduces the coagulation of blood, i.e. the coagulation cascade leading to fibrin polymerization and therefore fibrin clot formation. Anticoagulants thereby prolong the clotting time by inhibiting the coagulation cascade by clotting factors after the initial platelet aggregation.

As used herein, the term “anti-platelet agent” means a substance that decreases platelet aggregation and/or inhibits thrombus formation, i.e. a substance that inhibits the initial platelet aggregation of the blood clotting. Anti-platelet agents thus interfere with the platelet activation cascade leading to activated platelets which can adhere to fibrin fibers, other extracellular matrix components or aggregate into platelet aggregates. It is emphasized that the coagulation cascade and the platelet aggregation cascade are two separate cascades, even though some proteins, such as thrombin, may play a role in both cascades. Antiplatelet drugs can reversibly or irreversibly inhibit the process involved in platelet activation resulting in decreased tendency of platelets to adhere to one another and to damaged blood vessels endothelium or to foreign material surfaces, as e.g. a blood sampler material. An example of an anti-platelet agent is iloprost. Iloprost possesses 6 asymmetrical carbon atoms of which 5 are common with those in the natural prostacyclin. The configuration of the molecule is therefore namely 8S, 9S, 11R, 12S and 15S. The methyl group at C16 causes two isomers to occur: 16R and 16S. Consequently, iloprost consists of two optically active diastereoisomers. Iloprost also contains two defined configurations of carbon-carbon double bonds, 5E and 13E—this stereo-specific formation is common in prostacyclin chemistry. Another example is prostacyclin per se, which is also referred to as epoprostenol.

As used herein, the term “blood sample” or “blood analysis sample” refers to a sample of blood that is suitable for diagnostic or analytical purposes. Thus, the blood sample comprises a relatively low volume of blood (from 20 pL to 10 mL blood), i.e. not the volumes e.g. required for blood donations (up to about 450 mL blood). A “blood sample suitable for BG and BMP parameter analysis and platelet count” means that the blood sample is suitable for use in the determination of BG, BMP parameters as well as performing a platelet count, wherein the anticoagulant and/or the anti-platelet agent does not interfere or at least not substantially interfere with the determination of one of the parameters.

In the context of the present invention, the term “sugar” refers to a mono-, di-, or tri-saccharide. Examples of sugars in the context of the invention include glucose, raffinose, galactose, fructose, xylose, sucrose, lactose, maltose, isomaltulose, and trehalose.

In the context of the present invention, the term “matrix material” refers to the material used to form a matrix, wherein the anti-platelet agent is dispersed. The matrix material may be a water-soluble polymer, a sugar, or both.

When referring to molecular weights of polymers herein, unless anything else is specified, is referring to weight average molecular weight.

Water-Soluble Polymers

Solubility in water of the solid mixture is advantageous in view of its intended use for blood samples. A large number of water-soluble polymers are known e.g. in pharmaceutical applications, such as excipients for tablets etc. These are chemically inert and do not interfere with active compounds, such as prostacyclin or analogues thereof. Among the known water-soluble polymers suitable for use in the present invention are polyethylene oxide (PEO), PEO derivatives, poloxamers, poloxamines, polyvinylpyrrolidone (PVP), hydroxypropyl cellulose, hypromellose, hypromellose phthalate, hypromellose acetate succinate, polyacrylates, polymethacrylates, poly(isopropylacrylamide), polyacrylamide, polyethylene glycol (PEG), PEO/polypropylene glycol copolymers, PEG-modified starches, vinyl acetate-vinyl pyrrolidone copolymers, polyacrylic acid copolymers, polymethacrylic acid copolymers, plant proteins, protein hydrolysates, polyelectrolytes, polyvinyl alcohol, poly (2-oxazoline), polyethylenimine, cucurbit[n]uril hydrate, maleic anhydride copolymers, polyphosphates, polyphosphazenes, xanthan gum, pectins, chitosan derivatives, dextran, carrageenan, guar gum, cellulose ethers, hyaluronic acid, albumin, and starch and starch derivatives. Accordingly, in one embodiment, the water-soluble polymer is selected from PEO, PEO derivatives, poloxamers, poloxamines, PVP, hydroxypropyl cellulose, hypromellose, hypromellose phthalate, hypromellose acetate succinate, polyacrylates, polymethacrylates, poly(isopropylacrylamide), polyacrylamide, PEG, PEO/polypropylene glycol copolymers, PEG-modified starches, vinyl acetate-vinyl pyrrolidone copolymers, polyacrylic acid copolymers, polymethacrylic acid copolymers, plant proteins, protein hydrolysates, polyelectrolytes, polyvinyl alcohol, poly (2-oxazoline), polyethylenimine, cucurbit[n]uril hydrate, maleic anhydride copolymers, polyphosphates, polyphosphazenes, xanthan gum, pectins, chitosan derivatives, dextran, carrageenan, guar gum, cellulose ethers, hyaluronic acid, albumin, starch and starch derivatives, and mixtures thereof. In a further embodiment, the water-soluble polymer is selected from PEO, poloxamers, poloxamines, PVP, hydroxypropyl cellulose, hypromellose, hypromellose phthalate, hypromellose acetate succinate, polyacrylates, polymethacrylates, poly(isopropylacrylamide), polyacrylamide, PEG, PEO/polypropylene glycol copolymers, vinyl acetate-vinyl pyrrolidone copolymers, polyvinyl alcohol, poly (2-oxazoline), polyphosphates, polyphosphazenes, xanthan gum, pectins, dextran, carrageenan, guar gum, cellulose ethers, hyaluronic acid, starch and starch derivatives, and mixtures thereof. In still a further embodiment, the water-soluble polymer is selected from PEO, PVP, hydroxypropyl cellulose, hypromellose, polyacrylates, polymethacrylates, polyacrylamide, PEG, PEO/polypropylene glycol copolymers, polyvinyl alcohol, polyphosphates, xanthan gum, pectins, dextran, carrageenan, guar gum, starch and starch derivatives, and mixtures thereof. In yet a further embodiment, the water-soluble polymer is selected from PEO, PVP, hydroxypropyl cellulose, hypromellose, PEG, polyvinyl alcohol, xanthan gum, guar gum, starch and starch derivatives, and mixtures thereof. In another embodiment, the water-soluble polymer is selected from PEO, PVP, hydroxypropyl cellulose, hypromellose, PEG, polyvinyl alcohol, and mixtures thereof. In a further embodiment, the water-soluble polymer is selected from PVP, hydroxypropyl cellulose, hypromellose, and mixtures thereof. In still another embodiment, the water-soluble polymer is selected from PVP, hypromellose, and mixtures thereof. In yet another embodiment, the water-soluble polymer is PVP.

Water-soluble polymers are typically available with many different molecular weights, with lower molecular weights typically having higher water solubility and faster dissolution. In one embodiment, the water-soluble polymer has a molecular weight in the range 1000-450000 Da. In a further embodiment the water-soluble polymer has a molecular weight in the range 2000-100000 Da. In a further embodiment the water-soluble polymer has a molecular weight in the range 3000-60000 Da.

Prostacyclin Analogs

Prostacyclin and analogs thereof prevent formation of the platelet plug involved in primary hemostasis (a part of blood clot formation). They do this by inhibiting platelet activation.

They are also effective vasodilators. In the context of the present invention, prostacyclin analogs include iloprost, beraprost, treprostinil, cicaprost, carbacyclin, EP 157 ((Z)-7-[(1R,2R,3R,4S)-3-[(Z)-benzhydryloxyiminomethyl]-2-bicyclo[2.2.2]oct-5-enyl]hept-5-enoic acid), FK-788 (2-[[(6R)-6-(diphenylcarbamoyloxymethyl)-6-hydroxy-7,8-dihydro-5H-naphthalen-1-yl]oxy]acetic acid), and taprostene. These compounds may be either in the form of the free acid or in the form of a salt thereof. Furthermore, they exist in various stereoisomeric forms. Accordingly, the compound comprised in the solid mixture contained in the blood sampler according to the invention is selected from iloprost, beraprost, treprostinil, cicaprost, carbacyclin, EP 157 ((Z)-7-[(1R,2R,3R,4S)-3-[(Z)-benzhydryloxyiminomethyl]-2-bicyclo[2.2.2]oct-5-enyl]hept-5-enoic acid), FK-788 (2-[[(6R)-6-(diphenylcarbamoyloxymethyl)-6-hydroxy-7,8-dihydro-5H-naphthalen-1-yl]oxy]acetic acid), and taprostene, as well as any stereoisomers and salts thereof.

In one embodiment, the compound comprised in the solid mixture contained in the blood sampler according to the invention is selected from iloprost, beraprost, treprostinil, cicaprost, carbacyclin, and EP 157 ((Z)-7-[(1R,2R,3R,4S)-3-[(Z)-benzhydryloxyiminomethyl]-2-bicyclo[2.2.2]oct-5-enyl]hept-5-enoic acid), as well as any stereoisomers and salts thereof. In a further embodiment, the compound comprised in the solid mixture contained in the blood sampler according to the invention is selected from iloprost, beraprost, treprostinil, cicaprost, and carbacyclin, as well as any stereoisomers and salts thereof. In still a further embodiment, the compound comprised in the solid mixture contained in the blood sampler according to the invention is selected from iloprost, beraprost, and treprostinil, as well as any stereoisomers and salts thereof.

In another embodiment, the compound comprised in the solid mixture contained in the blood sampler according to the invention is iloprost or a salt of iloprost. In still another embodiment, the compound comprised in the solid mixture contained in the blood sampler according to the invention is a salt of iloprost, such as the trometamol salt. In yet another embodiment, the iloprost is the 8S, 9S, 11R, 12S, 16S-isomer of iloprost.

The skilled person will understand that the amount of prostacyclin analog should be in sufficient amounts to exert its anti-platelet effect on the blood sample to be loaded into the blood sampler. The skilled person will further understand that in practical applications, the amount of prostacyclin analogs will be limited due to economic considerations. Furthermore, without being bound by a particular theory, an amount that avoids a too high concentration, will assist in avoiding degradation products, such as the dimeric esters known for iloprost. In one embodiment, the amount of the compound comprised in the solid mixture, such as iloprost or a salt thereof, is in the range of 0.01 to 20.0% (w/w) of the solid mixture. In a further embodiment, the amount of the compound comprised in the solid mixture, such as iloprost or a salt thereof, is in the range of 0.05 to 5.0% (w/w) of the solid mixture, such as in the range of 0.1 to 2.5% of the solid mixture, e.g. in the range of 0.2 to 1.5% of the solid mixture. In another embodiment, the amount of the compound comprised in the solid mixture, such as iloprost or a salt thereof, is in the range of 0.5 to 10.0% (w/w) of the solid mixture, such as in the range of 0.7 to 4.0% of the solid mixture.

Anticoagulants

As discussed above, the “3-in-1” method includes the use of an anticoagulant in addition to the anti-platelet agent. The anticoagulant may be comprised in the blood sampler or it may be added separately. Accordingly, in one embodiment, the blood sampler further comprises an anticoagulant.

In a further embodiment, the anticoagulant comprised in the blood sampler is separate from the matrix of the water-soluble polymer material.

A number of anticoagulants are known to the skilled person. These include heparinates and heparinoids. Heparinates include heparin, such as unfractionated heparin, high molecular weight heparin (HMWH), low molecular weight heparin (LMWH), including bemiparin, certoparin, dalteparin, enoxaparin, nadroparin, pamaparin, reviparin, tinzaparin; and oligosaccharides such as fondaparinux and idraparinux. Heparinoids include danaparoid, dermatan sulfate and sulodexide.

In one embodiment, the anticoagulant is a heparinate selected from heparin, such as unfractionated heparin, high molecular weight heparin (HMWH), low molecular weight heparin (LMWH), including bemiparin, certoparin, dalteparin, enoxaparin, nadroparin, pamaparin, reviparin, tinzaparin; and oligosaccharides such as fondaparinux and idraparinux, as well as salts thereof. In another embodiment, the anticoagulant comprises heparin.

Heparin is a naturally occurring polysaccharide that inhibits coagulation, the process that leads to thrombosis. Natural heparin consists of molecular chains of varying lengths, or molecular weights. It is also used as an anticoagulant medication (blood thinner). It binds to the enzyme inhibitor antithrombin III (AT), causing a conformational change that results in its activation through an increase in the flexibility of its reactive site loop. The activated AT then inactivates thrombin, factor Xa and other proteases.

In one embodiment, the anticoagulant is electrolyte-balanced heparin, (also called “balanced heparin”). Heparin is known to bind positively charged electrolytes and this may interfere with electrolyte measurements. Formulations of electrolyte-balanced heparin may comprise lithium, zinc, sodium, potassium, calcium, magnesium and/or ammonium salts of heparin. In a further embodiment, the electrolyte-balanced heparin comprises sodium, potassium, calcium and/or magnesium salts of heparin.

In another embodiment, the heparin is human heparin, pig heparin or synthetic heparin. In still another embodiment, the heparin is pig heparin. In a further embodiment, the anticoagulant is unfractionated heparin.

In one embodiment, the anticoagulant can be in a liquid form, also called “liquid heparin”, or in a dry form, e.g. as dry balanced heparin. One example of a dry form of the anticoagulant is a lyophilized anticoagulant, e.g. lyophilized heparin or lyophilized balanced heparin. In a further embodiment, the anticoagulant is in lyophilized form.

Buffers and Sugars

Some of the prostacyclin analogs according to the present invention are sensitive to certain pH conditions. As an example, iloprost is sensitive to acidic pH. Hence, in some cases it may be beneficial to add a buffer to the solid mixture. Accordingly, in one embodiment, the solid mixture further comprises a buffer material. In a further embodiment, the buffer material is a Tris buffer.

Many different buffers are available and most pH values within the normal range of 0-14 may be achieved with a buffer. In some cases, such as in the case of iloprost, a buffer providing an alkaline pH is beneficial. Thus, in one embodiment, the buffer provides a pH in the range of 7.5 to 12.0. In a further embodiment, the buffer provides a pH in the range of 7.6 to 11.0. In still a further embodiment, the buffer provides a pH in the range of 7.7 to 10.0. In yet a further embodiment, the buffer provides a pH in the range of 7.8 to 9.5. In another embodiment, the buffer provides a pH in the range of 7.9 to 9.0.

Without being bound by any theory, it is further contemplated that some prostacyclin analogs may benefit from increased stability by including a sugar in the solid mixture. Accordingly, in one embodiment, the solid mixture further comprises a sugar dispersed in the matrix of the water-soluble polymer. In another embodiment, the sugar is selected from the group consisting of glucose, raffinose, galactose, fructose, xylose, sucrose, lactose, maltose, isomaltulose, trehalose, and mixtures thereof. In still another embodiment, the sugar is selected from the group consisting of glucose, raffinose, sucrose, and mixtures thereof.

It has also been found that a sugar may result in a water-soluble matrix on its own and provide solubility increases at a similar level as the water-soluble polymers defined herein. Accordingly, in one aspect, the present invention concerns a blood sampler containing a solid mixture comprising a compound selected from iloprost, beraprost, treprostinil, cicaprost, carbacyclin, EP 157 ((Z)-7-[(1R,2R,3R,4S)-3-[(Z)-benzhydryloxyiminomethyl]-2-bicyclo[2.2.2]oct-5-enyl]hept-5-enoic acid), FK-788 (2-[[(6R)-6-(diphenylcarbamoyloxymethyl)-6-hydroxy-7,8-dihydro-5H-naphthalen-1-yl]oxy]acetic acid), and taprostene, as well as any stereoisomers and salts thereof, wherein said compound is dispersed in a matrix of a sugar. In one embodiment, the sugar is selected from the group consisting of glucose, raffinose, galactose, fructose, xylose, sucrose, lactose, maltose, isomaltulose, trehalose, and mixtures thereof. In another embodiment, the sugar is selected from the group consisting of glucose, raffinose, sucrose, and mixtures thereof.

In one embodiment, the matrix material comprises a sugar and less than 5% by weight of the total matrix material of a water-soluble polymer, such as less than 2% by weight of the total matrix material, e.g. less than 1% by weight of the total matrix material. In another embodiment, the matrix material comprises only a sugar and no water-soluble polymer.

In a further embodiment, the matrix material comprises a water-soluble polymer and less than 5% by weight of the total matrix material of a sugar, such as less than 2% by weight of the total matrix material, e.g. less than 1% by weight of the total matrix material. In still a further embodiment, the matrix material comprises only a water-soluble polymer and no sugar.

In yet another embodiment, the matrix material comprises both a water-soluble polymer and a sugar. In a further embodiment, the matrix material comprises the water-soluble polymer and the sugar in a weight ratio of 70:30 to 30:70, such as in the weight ratio 60:40 to 40:60, e.g. in the weight ratio 50:50.

### Further Embodiments

For each of the features of the present invention discussed above, specific embodiments are disclosed. It is further contemplated that the present invention may by directed at combinations of these embodiments. Hence, in one embodiment, the water-soluble polymer is selected from PEO, PVP, hydroxypropyl cellulose, hypromellose, PEG, polyvinyl alcohol, and mixtures thereof, and the compound comprised in the solid mixture contained in the blood sampler according to the invention is selected from iloprost, beraprost, treprostinil, cicaprost, carbacyclin, and EP 157 ((Z)-7-[(1R,2R,3R,4S)-3-[(Z)-benzhydryloxyiminomethyl]-2-bicyclo[2.2.2]oct-5-enyl]hept-5-enoic acid), as well as any stereoisomers and salts thereof. In a further embodiment, the water-soluble polymer is selected from PEO, PVP, hydroxypropyl cellulose, hypromellose, PEG, polyvinyl alcohol, and mixtures thereof, and the compound comprised in the solid mixture contained in the blood sampler according to the invention is selected from iloprost, beraprost and treprostinil, as well as any stereoisomers and salts thereof. In still a further embodiment, the water-soluble polymer is selected from PEO, PVP, hydroxypropyl cellulose, hypromellose, PEG, polyvinyl alcohol, and mixtures thereof, and the compound comprised in the solid mixture contained in the blood sampler according to the invention is iloprost or a salt of iloprost.

In another embodiment, the water-soluble polymer is selected from PVP, hypromellose, and mixtures thereof, and the compound comprised in the solid mixture contained in the blood sampler according to the invention is selected from iloprost, beraprost, treprostinil, cicaprost, carbacyclin, and EP 157 ((Z)-7-[(1R,2R,3R,4S)-3-[(Z)-benzhydryloxyiminomethyl]-2-bicyclo[2.2.2]oct-5-enyl]hept-5-enoic acid), as well as any stereoisomers and salts thereof. In still another embodiment, the water-soluble polymer is selected from PVP, hypromellose, and mixtures thereof, and the compound comprised in the solid mixture contained in the blood sampler according to the invention is selected from iloprost, beraprost and treprostinil, as well as any stereoisomers and salts thereof. In yet another embodiment, the water-soluble polymer is selected from PVP, hypromellose, and mixtures thereof, and the compound comprised in the solid mixture contained in the blood sampler according to the invention is iloprost or a salt of iloprost.

In a further embodiment, the water-soluble polymer is PVP, and the compound comprised in the solid mixture contained in the blood sampler according to the invention is selected from iloprost, beraprost, treprostinil, cicaprost, carbacyclin, and EP 157 ((Z)-7-[(1R,2R,3R,4S)-3-[(Z)-benzhydryloxyiminomethyl]-2-bicyclo[2.2.2]oct-5-enyl]hept-5-enoic acid), as well as any stereoisomers and salts thereof. In still a further embodiment, the water-soluble polymer is PVP, and the compound comprised in the solid mixture contained in the blood sampler according to the invention is selected from iloprost, beraprost, and treprostinil, as well as any stereoisomers and salts thereof. In yet a further embodiment, the water-soluble polymer is PVP, and the compound comprised in the solid mixture contained in the blood sampler according to the invention is iloprost or a salt of iloprost.

In another embodiment, the water-soluble polymer is selected from PEO, PVP, hydroxypropyl cellulose, hypromellose, PEG, polyvinyl alcohol, and mixtures thereof, and the compound comprised in the solid mixture contained in the blood sampler according to the invention is selected from iloprost, beraprost, treprostinil, cicaprost, carbacyclin, and EP 157 ((Z)-7-[(1R,2R,3R,4S)-3-[(Z)-benzhydryloxyiminomethyl]-2-bicyclo[2.2.2]oct-5-enyl]hept-5-enoic acid), as well as any stereoisomers and salts thereof, in an amount in the range of 0.05 to 5.0% by weight of the solid mixture. In still another embodiment, the water-soluble polymer is selected from PEO, PVP, hydroxypropyl cellulose, hypromellose, PEG, polyvinyl alcohol, and mixtures thereof, and the compound comprised in the solid mixture contained in the blood sampler according to the invention is selected from iloprost, beraprost, and treprostinil, as well as any stereoisomers and salts thereof, in an amount in the range of 0.05 to 5.0% by weight of the solid mixture. In yet another embodiment, the water-soluble polymer is selected from PEO, PVP, hydroxypropyl cellulose, hypromellose, PEG, polyvinyl alcohol, and mixtures thereof, and the compound comprised in the solid mixture contained in the blood sampler according to the invention is iloprost or a salt of iloprost in an amount in the range of 0.05 to 5.0% by weight of the solid mixture.

In a further embodiment, the water-soluble polymer is selected from PVP, hypromellose, and mixtures thereof, and the compound comprised in the solid mixture contained in the blood sampler according to the invention is selected from iloprost, beraprost, treprostinil, cicaprost, carbacyclin, and EP 157 ((Z)-7-[(1R,2R,3R,4S)-3-[(Z)-benzhydryloxyiminomethyl]-2-bicyclo[2.2.2]oct-5-enyl]hept-5-enoic acid), as well as any stereoisomers and salts thereof, in an amount in the range of 0.05 to 5.0% by weight of the solid mixture. In still a further embodiment, the water-soluble polymer is selected from PVP, hypromellose, and mixtures thereof, and the compound comprised in the solid mixture contained in the blood sampler according to the invention is selected from iloprost, beraprost, and treprostinil, as well as any stereoisomers and salts thereof, in an amount in the range of 0.05 to 5.0% by weight of the solid mixture. In yet a further embodiment, the water-soluble polymer is selected from PVP, hypromellose, and mixtures thereof, and the compound comprised in the solid mixture contained in the blood sampler according to the invention is iloprost or a salt of iloprost in an amount in the range of 0.05 to 5.0% by weight of the solid mixture.

In another embodiment, the water-soluble polymer is PVP, and the compound comprised in the solid mixture contained in the blood sampler according to the invention is selected from iloprost, beraprost, treprostinil, cicaprost, carbacyclin, and EP 157 ((Z)-7-[(1R,2R,3R,4S)-3-[(Z)-benzhydryloxyiminomethyl]-2-bicyclo[2.2.2]oct-5-enyl]hept-5-enoic acid), as well as any stereoisomers and salts thereof, in an amount in the range of 0.05 to 5.0% by weight of the solid mixture. In still another embodiment, the water-soluble polymer is PVP, and the compound comprised in the solid mixture contained in the blood sampler according to the invention is selected from iloprost, beraprost, and treprostinil, as well as any stereoisomers and salts thereof, in an amount in the range of 0.05 to 5.0% by weight of the solid mixture. In yet another embodiment, the water-soluble polymer is PVP, and the compound comprised in the solid mixture contained in the blood sampler according to the invention is iloprost or a salt of iloprost in an amount in the range of 0.05 to 5.0% by weight of the solid mixture.

In a further embodiment, the water-soluble polymer is selected from PEO, PVP, hydroxypropyl cellulose, hypromellose, PEG, polyvinyl alcohol, and mixtures thereof, and the compound comprised in the solid mixture contained in the blood sampler according to the invention is selected from iloprost, beraprost, treprostinil, cicaprost, carbacyclin, and EP 157 ((Z)-7-[(1R,2R,3R,4S)-3-[(Z)-benzhydryloxyiminomethyl]-2-bicyclo[2.2.2]oct-5-enyl]hept-5-enoic acid), as well as any stereoisomers and salts thereof, in an amount in the range of 0.1 to 2.5% by weight of the solid mixture. In still a further embodiment, the water-soluble polymer is selected from PEO, PVP, hydroxypropyl cellulose, hypromellose, PEG, polyvinyl alcohol, and mixtures thereof, and the compound comprised in the solid mixture contained in the blood sampler according to the invention is selected from iloprost, beraprost, and treprostinil, as well as any stereoisomers and salts thereof, in an amount in the range of 0.1 to 2.5% by weight of the solid mixture. In yet a further embodiment, the water-soluble polymer is selected from PEO, PVP, hydroxypropyl cellulose, hypromellose, PEG, polyvinyl alcohol, and mixtures thereof, and the compound comprised in the solid mixture contained in the blood sampler according to the invention is iloprost or a salt of iloprost in an amount in the range of 0.1 to 2.5% by weight of the solid mixture.

In a further embodiment, the water-soluble polymer is selected from PVP, hypromellose, and mixtures thereof, and the compound comprised in the solid mixture contained in the blood sampler according to the invention is selected from iloprost, beraprost, treprostinil, cicaprost, carbacyclin, and EP 157 ((Z)-7-[(1R,2R,3R,4S)-3-[(Z)-benzhydryloxyiminomethyl]-2-bicyclo[2.2.2]oct-5-enyl]hept-5-enoic acid), as well as any stereoisomers and salts thereof, in an amount in the range of 0.1 to 2.5% by weight of the solid mixture. In still a further embodiment, the water-soluble polymer is selected from PVP, hypromellose, and mixtures thereof, and the compound comprised in the solid mixture contained in the blood sampler according to the invention is selected from iloprost, beraprost, and treprostinil, as well as any stereoisomers and salts thereof, in an amount in the range of 0.1 to 2.5% by weight of the solid mixture. In yet a further embodiment, the water-soluble polymer is selected from PVP, hypromellose, and mixtures thereof, and the compound comprised in the solid mixture contained in the blood sampler according to the invention is iloprost or a salt of iloprost in an amount in the range of 0.1 to 2.5% by weight of the solid mixture.

In another embodiment, the water-soluble polymer is PVP, and the compound comprised in the solid mixture contained in the blood sampler according to the invention is selected from iloprost, beraprost, treprostinil, cicaprost, carbacyclin, and EP 157 ((Z)-7-[(1R,2R,3R,4S)-3-[(Z)-benzhydryloxyiminomethyl]-2-bicyclo[2.2.2]oct-5-enyl]hept-5-enoic acid), as well as any stereoisomers and salts thereof, in an amount in the range of 0.1 to 2.5% by weight of the solid mixture. In still another embodiment, the water-soluble polymer is PVP, and the compound comprised in the solid mixture contained in the blood sampler according to the invention is selected from iloprost, beraprost, and treprostinil, as well as any stereoisomers and salts thereof, in an amount in the range of 0.1 to 2.5% by weight of the solid mixture. In yet another embodiment, the water-soluble polymer is PVP, and the compound comprised in the solid mixture contained in the blood sampler according to the invention is iloprost or a salt of iloprost in an amount in the range of 0.1 to 2.5% by weight of the solid mixture.

In another embodiment, the water-soluble polymer is selected from PEO, PVP, hydroxypropyl cellulose, hypromellose, PEG, polyvinyl alcohol, and mixtures thereof, and the compound comprised in the solid mixture contained in the blood sampler according to the invention is selected from iloprost, beraprost, treprostinil, cicaprost, carbacyclin, and EP 157 ((Z)-7-[(1R,2R,3R,4S)-3-[(Z)-benzhydryloxyiminomethyl]-2-bicyclo[2.2.2]oct-5-enyl]hept-5-enoic acid), as well as any stereoisomers and salts thereof, in an amount in the range of 0.2 to 1.5% by weight of the solid mixture. In still another embodiment, the water-soluble polymer is selected from PEO, PVP, hydroxypropyl cellulose, hypromellose, PEG, polyvinyl alcohol, and mixtures thereof, and the compound comprised in the solid mixture contained in the blood sampler according to the invention is selected from iloprost, beraprost, and treprostinil, as well as any stereoisomers and salts thereof, in an amount in the range of 0.2 to 1.5% by weight of the solid mixture. In yet another embodiment, the water-soluble polymer is selected from PEO, PVP, hydroxypropyl cellulose, hypromellose, PEG, polyvinyl alcohol, and mixtures thereof, and the compound comprised in the solid mixture contained in the blood sampler according to the invention is iloprost or a salt of iloprost in an amount in the range of 0.2 to 1.5% by weight of the solid mixture.

In a further embodiment, the water-soluble polymer is selected from PVP, hypromellose, and mixtures thereof, and the compound comprised in the solid mixture contained in the blood sampler according to the invention is selected from iloprost, beraprost, treprostinil, cicaprost, carbacyclin, and EP 157 ((Z)-7-[(1R,2R,3R,4S)-3-[(Z)-benzhydryloxyiminomethyl]-2-bicyclo[2.2.2]oct-5-enyl]hept-5-enoic acid), as well as any stereoisomers and salts thereof, in an amount in the range of 0.2 to 1.5% by weight of the solid mixture. In still a further embodiment, the water-soluble polymer is selected from PVP, hypromellose, and mixtures thereof, and the compound comprised in the solid mixture contained in the blood sampler according to the invention is selected from iloprost, beraprost, and treprostinil, as well as any stereoisomers and salts thereof, in an amount in the range of 0.2 to 1.5% by weight of the solid mixture. In yet a further embodiment, the water-soluble polymer is selected from PVP, hypromellose, and mixtures thereof, and the compound comprised in the solid mixture contained in the blood sampler according to the invention is iloprost or a salt of iloprost in an amount in the range of 0.2 to 1.5% by weight of the solid mixture.

In another embodiment, the water-soluble polymer is PVP, and the compound comprised in the solid mixture contained in the blood sampler according to the invention is selected from iloprost, beraprost, treprostinil, cicaprost, carbacyclin, and EP 157 ((Z)-7-[(1R,2R,3R,4S)-3-[(Z)-benzhydryloxyiminomethyl]-2-bicyclo[2.2.2]oct-5-enyl]hept-5-enoic acid), as well as any stereoisomers and salts thereof, in an amount in the range of 0.2 to 1.5% by weight of the solid mixture. In still another embodiment, the water-soluble polymer is PVP, and the compound comprised in the solid mixture contained in the blood sampler according to the invention is selected from iloprost, beraprost, and treprostinil, as well as any stereoisomers and salts thereof, in an amount in the range of 0.2 to 1.5% by weight of the solid mixture. In yet another embodiment, the water-soluble polymer is PVP, and the compound comprised in the solid mixture contained in the blood sampler according to the invention is iloprost or a salt of iloprost in an amount in the range of 0.2 to 1.5% by weight of the solid mixture.

Preparation of the Solid Mixtures

The solid mixtures contained in the blood sampler according to the invention may be prepared by dissolving the compound and the water-soluble polymer in a suitable solvent, such as water or ethanol, by stirring and letting the solution dry. The solid mixtures may also be prepared spray drying of the solution containing the compound and the water-soluble polymer. A particularly useful spray drying technology is ultrasonic spray coating. In ultrasonic spray coating, ultrasound is used to generate atomization of the liquid. A small airflow is used to direct the spray mist away from the nozzle to the desired surface. Very small amounts of liquid can be applied in a reproducible way. This technique is useful for spray coating the inner surface of blood samplers, such as tubes and syringes. The spray nozzle goes into the blood sampler and sprays as the nozzle is withdrawn from the sampler. The atomized spray has a narrower drop size distribution than conventional pressure driven atomization which adds to the reproducibility of the spraying. Ultrasonic spray coating equipment is available inter alia from the company Sono-Tek, e.g. the ExactaCoat® coating system.

### EXAMPLES

Preparation of Solutions

A. 1 mL of a 5 mg/mL Iloprost solution in methyl acetate ex. Cayman Chemical is diluted with ethanol in a 25 mL measuring flask to get a solution concentration of 0.2 mg/mL.

B. A 25 mM Tris-solution is made by dissolving 0.07571 g of Tris(hydroxymethyl)aminomethane, Merck no. 1.08382.0500 in Milli-Q water in a 25 mL measuring flask.

C. A PVP-Tris solution is made by in a 50 mL measuring flask by dissolving 1.166 g of PVP-10 ex. Merck (Mw 10,000) in approx. 35 mL of Milli-Q water, adding 7.292 mL of 25 mM Tris-solution (B) and filling to the 50 mL mark with Milli-Q water.

D. The final solution is made by mixing 1442 μL of Iloprost solution (A) with 1224 μL of PVP-Tris solution (C).

When testing other matrix polymers and sugars, PVP is substituted on a mass basis in the PVP-Tris solution (C). For mixtures of polymers and sugars, equal volumes polymer-Tris and sugar-Tris solutions (612 μL of each) substitute the PVP-Tris solution. For water-soluble polymers the following products have been used: HPMC (Merck, Prod. no. H8384), PVP (Mw 10′000), Kolliphor P407 (Merck, Prod. no. 62035), PEG 10′000 (Merck, Prod. No. 92897), PEG 35′000 (Merck, Prod. No. 81310) and PVP 40′000 (Merck, Prod. No. PVP40). For sugars the following products have been used: D-(−)-fructose (Merck, Prod. No. F3510), D-(+)-galactose (Merck, Prod. No. G5388), glucose (Merck), raffinose (Merck, Prod. No. R0514) and sucrose (Merck, Prod. No. S7903).

When testing other anti-thrombotics, substitutions are made to solution A to achieve the same molar concentration. Beraprost sodium ex. Merck was first diluted in saline to 10 mM solution and then diluted further in 30% ethanol in water to achieve a solution of same molarity as Iloprost in A. Treprostinil ex. Merck was dissolved directly in ethanol.

Adding 10 μL of the final solution (D) to a blood collection tube, drying it and adding 3 mL of Milli-Q water theoretically gives an Iloprost concentration of 1000 nM.

Preparation of Tubes

3 mL blood collection tubes (No additives tube, Greiner Bio-one, Ref.no 454001) were used for testing.

For storage stability and whole blood stabilization testing, the tubes were sprayed with 10 μL each of the mixture (D) using an ExactaCoat from Sono-Tek for ultrasonic spraying. And tubes were evacuated after spraying in order to allow filling of 3 mL whole blood during blood drawing via venipuncture.

For dissolution tests, 10 μL of the solution mixture (D) was pipetted into the blood collection tubes and left to dry at least overnight.

Dissolution Testing

A 2 mm by 7 mm rounded PTFE coated magnet was added to each blood collection tube, which was then placed vertically in a holder on a magnetic stirrer at 920 rpm. At time 0, 3 mL of Milli-Q water was added, and at times 10 sec, 60 sec, and 300 sec, 100 μL was extracted from the blood collection tube and the Iloprost concentration determined on an HPLC-MS comparing to a standard curve. Beraprost and Treprostinil were compared to an Iloprost standard curve, therefore Beraprost and Treprostinil concentrations are only estimates.

### Example 1—Storage Stability

Closed tubes were closed prior to exposing them to the test environment, open tubes were stored together with their lids in an exicator together with a saturated solution of NaCl in Milli-Q water to obtain a 75% RH atmosphere (except for −18° C.) and stored at the indicated temperature. At the indicated storage time, 3 mL of Milli-Q water was added to each tube. Tubes were turned or shaken at least 3 times to ensure proper dissolution during at least 10 minutes. The concentration of Iloprost was measured on a HPLC-MS comparing to a curve made from standard solutions between 1 nM and 1500 nM of Iloprost.

Test tubes prepared as described above for a mixture of Iloprost, PVP, and Tris were stored as open or closed tubes at −18° C., 5° C., room temperature or 32° C. The relative Iloprost concentration compared to the concentration at the tube production (time 0, t0) was measured after approximately 1 week, 1 month, 2 months and 3 months.

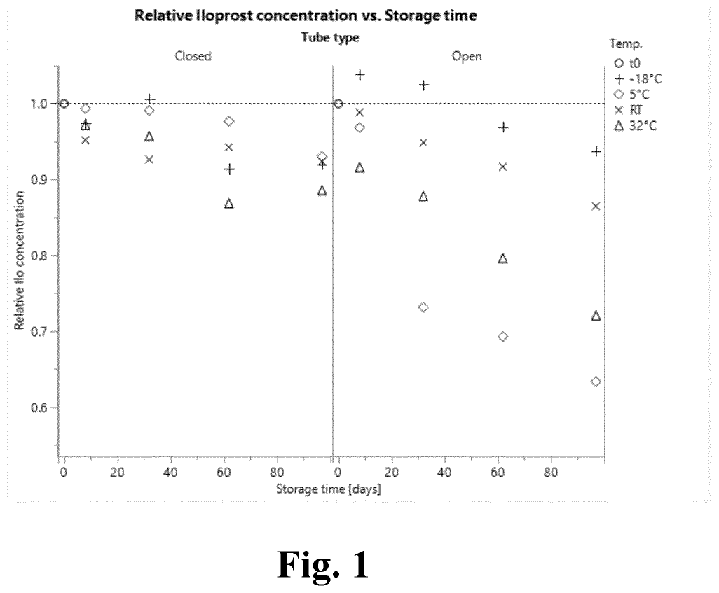

The results are shown in FIG. 1. They show that stability is highest at −18° C., but that it is also fairly high at 32° C., in particular in closed tubes.

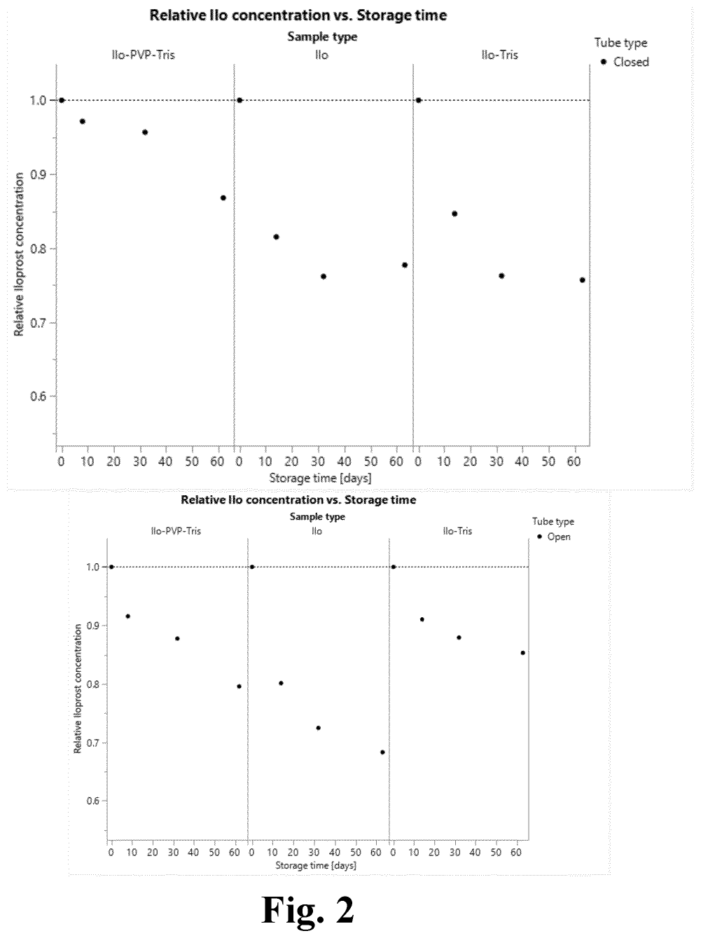

The relative Iloprost concentration in comparison to Iloprost concentration at tube production (time 0) was also determined for Iloprost/PVP/Tris sprayed blood collection tubes in comparison to Iloprost/Tris and Iloprost (non-formulated) either open or closed stored at a temperature of 32° C. for approx. 1 week, 1 month and 2 months. The results are shown in FIG. 2. They show that stability is increased by the PVP matrix, and that Tris also contributes to stability.

### Example 2—Dissolution Rate with and without Matrix

The effect of Iloprost formulation on achieving fast dissolution of Iloprost was tested. Iloprost concentrations extracted from dried formulated Iloprost/PVP(10′000)/Tris, versus Iloprost/Tris and Iloprost in blood collection tubes were determined. Iloprost concentrations were determined at extraction times of 10 seconds, 1 minute and 5 minutes of n=4 replicate tubes.

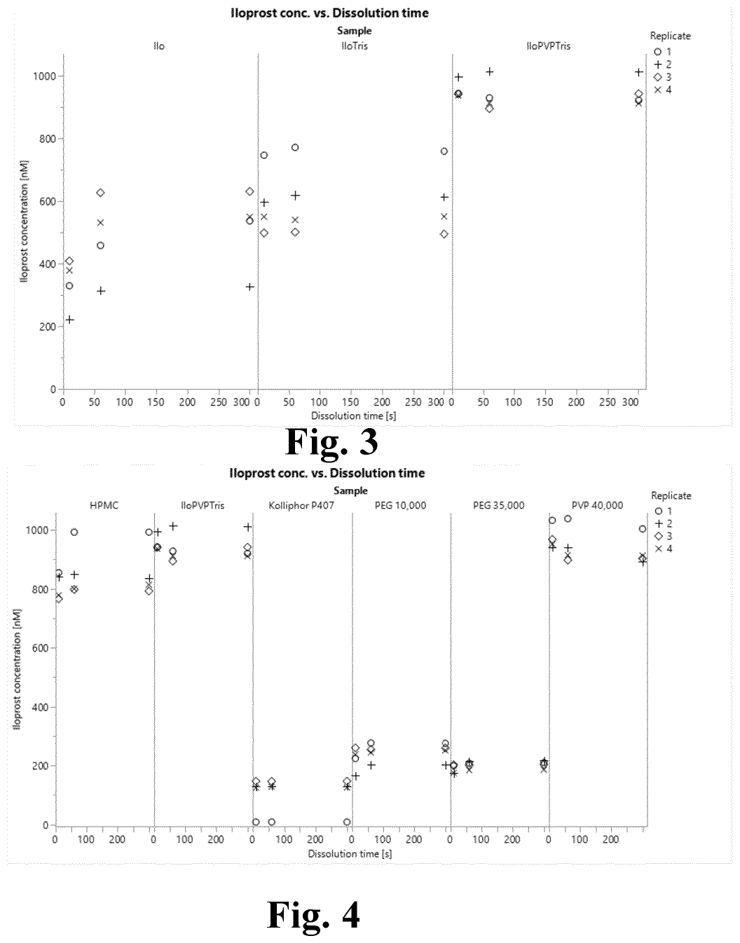

The results are shown in FIG. 3. They demonstrate that the dissolution rate is increased significantly with the PVP matrix.

### Example 3—Dissolution Rate with Different Polymers

The effect of different water-soluble polymers on the dissolution rate of Iloprost was tested. Iloprost concentrations extracted from tubes with dried formulated Iloprost/polymer/Tris were determined for polymers HPMC, PVP (Mw 10′000; labelled IloPVPTris), Kolliphor P407, PEG 10′000, PEG 35′000 and PVP 40′000. Iloprost concentrations were determined for extraction times of 10 seconds, 1 minute and 5 minutes of n=4 replicate tubes.

The results are shown in FIG. 4. They demonstrate that the different molecular weight PVP polymers work equally well. They furthermore demonstrate that cellulose derivatives, such as HPMC, provide a similar level of dissolution.

### Example 4—Dissolution Rate with Different Sugars

The effect of sugars used in Iloprost formulation on the dissolution rate of dried formulated Iloprost was tested. Iloprost concentrations extracted from tubes with dried formulated Iloprost/sugar/Tris were determined for sugars fructose (IloFruTris), galactose (IloGalTris), glucose (IloGluTris), raffinose (IloRafTris) and sucrose (IloSucTris). Iloprost concentrations were determined for extraction times of 10 seconds, 1 minute and 5 minutes of n=3 or 4 replicate tubes.

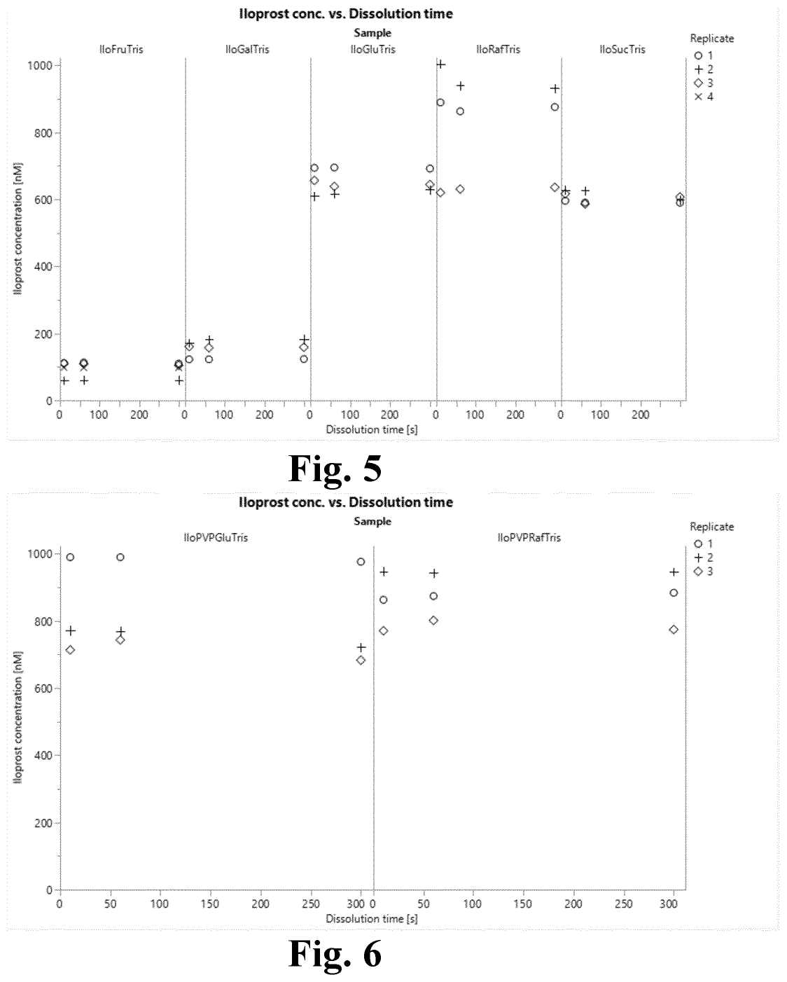

The results are shown in FIG. 5. They demonstrate that sugars on their own may achieve dissolution similar to dissolution obtained using water-soluble polymers.

The effect of polymer and sugar mixtures used in Iloprost formulation on the dissolution rate of dried formulated Iloprost was also tested. Iloprost concentrations extracted from tubes with dried formulated Iloprost/polymer+sugar mixture/Tris were determined for PVP (10′000) and glucose mixture (IloPVPGIuTris) and PVP (10′000) and raffinose mixture (IloPVPRafTris). Iloprost concentrations were determined for extraction times of 10 seconds, 1 minute and 5 minutes of n=3 replicate tubes.

The results are shown in FIG. 6. They demonstrate that replacing half the PVP with either of glucose or raffinose provides similar dissolution.

### Example 5—Dissolution Rate of Other Anti-Platelet Agents

The dissolution rate of dried formulated Beraprost sodium, Iloprost and Treprostinil was tested. Anti-platelet agent concentrations, as estimated from and Iloprost concentration standard curve, extracted from tubes with dried formulated anti-platelet agent Beraprost (BerPVPTris), Iloprost (IloPVPTris) and Treprostinil (TrePVPTris) were determined. Concentrations were determined for extraction times of 10 seconds, 1 minute and 5 minutes of n=4 or 5 replicate tubes.

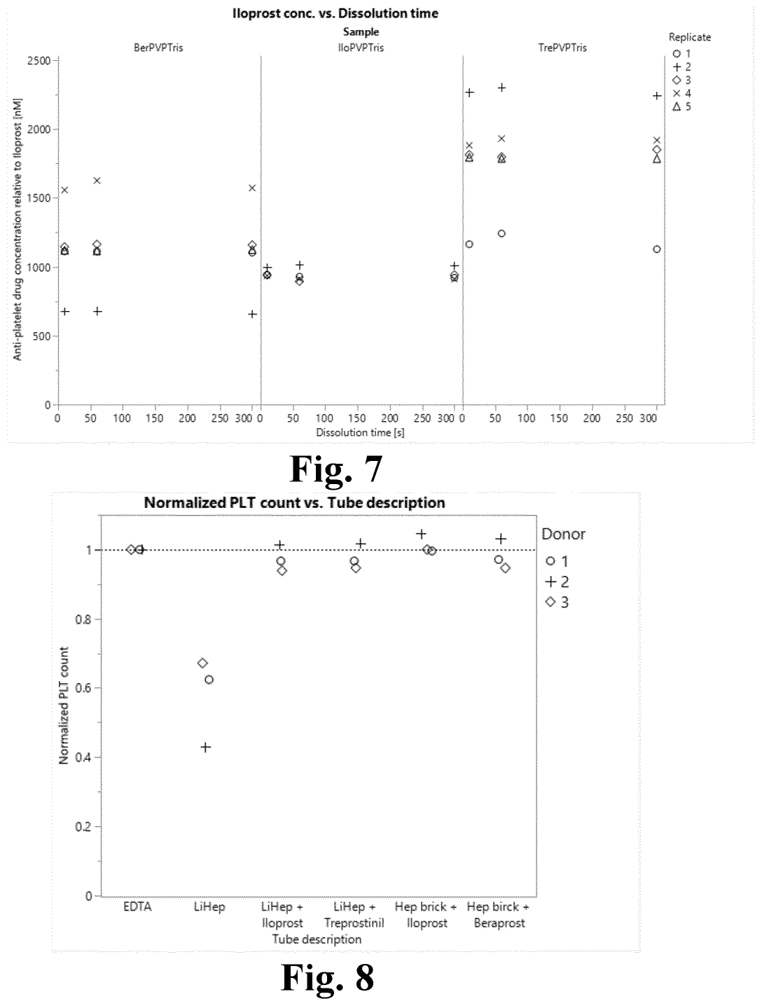

The results are shown in FIG. 7. They demonstrate that dissolution of matrix formulations of Beraprost and Treprostinil is achieved at a similar level to Iloprost.

### Example 6—Whole Blood Stability

Tubes with sprayed anti-platelet agents Iloprost, Beraprost sodium and Treprostinil each formulated with a PVP (Mw 10′000)/Tris matrix were prepared by ultrasonic spraying as described above. Spraying was done into either 1) tubes with no additive and after spraying, a heparinized filter paper brick (Hep brick) was added to the tube; or 2) spraying was conducted into commercial Lithium Heparin tubes (LiHep). The formulations were prepared to achieve a final anti-platelet agent concentration of 1 μM in whole blood. After preparation, tubes were evacuated and lids closed in order to allow drawing of correct whole blood volume during venipuncture. Blood from three different healthy volunteers were drawn into prepared blood collection tubes and reference standard EDTA and Lithium Heparin (LiHep) tubes. Whole blood samples were analyzed with a Sysmex XN-9000 hematology analyzer within 6 hours after blood sample drawing.

The results are shown in FIG. 8. The Y-axis is the normalized PLT concentration relative to the EDTA sample. The results demonstrate that, unlike heparin alone, the anti-platelet activity of Iloprost, Treprostinil, and Beraprost is as high as that of EDTA. Hence, in addition to increasing the stability and dissolution rate of anti-platelet agents, the matrix formulations of the invention do not interfere with the anti-platelet activity.

1. A blood sampler containing a solid mixture comprising a compound selected from iloprost, beraprost, treprostinil, cicaprostcarbacyclin, EP 157 ((Z)-7-[(1R,2R,3R,4S)-3-[(Z)-benzhydryloxyiminomethyl]-2-bicyclo[2.2.2]oct-5-enyl]hept-5-enoic acid), FK-788 (2-[[(6R)-6-(diphenylcarbamoyloxymethyl)-6-hydroxy-7,8-dihydro-5H-naphthalen-1-yl]oxy]acetic acid), and taprostene, a stereoisomer of any of the foregoing, and a salts of any of the foregoing, wherein said compound, stereoisomer, or salt is dispersed in a matrix of a water-soluble polymer and/or a sugar.

2. The blood sampler according to claim 1, wherein the blood sampler is selected from a syringe, a capillary, a test tube, and a cuvette.

3. The blood sampler according to claim 1, wherein the water-soluble polymer is selected from polyethylene oxide (PEO), PEO derivatives, poloxamers, poloxamines, polyvinylpyrrolidone (PVP), hydroxypropyl cellulose, hypromellose, hypromellose phthalate, hypromellose acetate succinate, polyacrylates, polymethacrylates, poly(isopropylacrylamide), polyacrylamide, polyethylene glycol (PEG), PEO/polypropylene glycol copolymers, PEG-modified starches, vinyl acetate-vinyl pyrrolidone copolymers, polyacrylic acid copolymers, polymethacrylic acid copolymers, plant proteins, protein hydrolysates, polyelectrolytes, polyvinyl alcohol, poly(2-oxazoline), polyethylenimine, cucurbit[n]uril hydrate, maleic anhydride copolymers, polyphosphates, polyphosphazenes, xanthan gum, pectins, chitosan derivatives, dextran, carrageenan, guar gum, cellulose ethers, hyaluronic acid, albumin, starch and starch derivatives, and mixtures thereof.

4. The blood sampler according to claim 1, wherein the water-soluble polymer is selected from PVP, hydroxypropyl cellulose, and hypromellose.

5. The blood sampler according to claim 3, wherein the water-soluble polymer is selected from PVP, hypromellose, and mixtures thereof.

6. The blood sampler according to claim 3, wherein the water-soluble polymer is PVP.

7. The blood sampler according to claim 1, wherein the water-soluble polymer has a molecular weight in the range of from 1000 to 450000 Da.

8. The blood sampler according to claim 1 comprising a compound selected from iloprost, beraprost, and treprostinil, a stereoisomer of any of the foregoing, and a salt of any of the foregoing thereof.

9. The blood sampler according to claim 1 comprising iloprost or a salt of iloprost.

10. The blood sampler according to claim 1, wherein the iloprost is the 8S, 9S, 11R, 12S, 16S-isomer of iloprost.

11. The blood sampler according to claim 1, wherein the amount of the compound in the solid mixture is in the range of from 0.01 to 20.0% (w/w) of the solid mixture.

12. The blood sampler according to claim 8, wherein the amount of the compound in the solid mixture is in the range of from 0.05 to 5.0% (w/w) of the solid mixture.

13. The blood sampler according to claim 1, wherein the solid mixture further comprises a buffer material.

14. The blood sampler according to claim 13, wherein the buffer material is a Tris buffer.

15. The blood sampler according to claim 1 further containing heparin or a salt thereof.

16. The blood sampler according to claim 15, wherein the heparin or salt thereof is electrolyte-balanced heparin.

17. The blood sampler according to claim 15, wherein the heparin or salt thereof is separate from the matrix of the water-soluble polymer material.

18. The blood sampler according to claim 1, wherein the solid mixture further comprises a sugar dispersed in the matrix of the water-soluble polymer.

19. The blood sampler according to claim 1, wherein the matrix material comprises a water-soluble polymer and less than 5% by weight of the total matrix material of a sugar.

20. The blood sampler according to claim 1, wherein the matrix material comprises a sugar and less than 5% by weight of the total matrix material of a water-soluble polymer.
